(19) United States
(12) Patent Application Publication
(10) Pub. No.: US 2016/0242792 A1
(43) Pub. Date: Aug. 25, 2016

(54) A DEVICE FOR INSERTING A SURGICAL PIN INTO A BONE STRUCTURE

(71) Applicant: ORTHOPAEDIC INTERNATIONAL, INC., Cabuyao, Laguna (PH)

(72) Inventors: Jude L. SASING, Quezon City (PH); Maynard T. LAPENA, Binangonan, Rizal (PH)

(21) Appl. No.: 15/030,248

(22) PCT Filed: Oct. 16, 2013
(86) PCT No.: PCT/IB2013/059392

§ 371 Date: Apr. 18, 2016

(51) Int. Cl. A61B 17/16; A61B 17/84; A61B 17/86; A61B 17/88

(52) U.S. Cl. CPC A61B 17/1697; A61B 17/162; A61B 17/8897; A61B 17/846; A61B 17/86; A61B 17/1615

(57) ABSTRACT

A device suitable for use in inserting a pin into an object comprises an elongated pin driver and an elongated pin which are engaged by a locking mechanism. The locking mechanism moves the device to a first locking position when the pin is inserted into the pin driver such that each of first and third abutment surfaces associated with a first abutting member of the pin driver and with a stud portion of the pin, respectively, abuts against one another, and such that each of second and fourth abutment surfaces associated with the first abutting member of the pin driver and with a second abutting member of the pin, respectively, abuts against one another in order to prevent the pin driver and the pin from rotating relative to each other in a first direction. The locking mechanism further moves the device to a second locking position when the pin driver and the pin are rotated relative to each other in a second direction substantially opposite the first direction until each of second and fifth abutment surfaces associated with the first abutting member of the pin driver and with a slot in the pin, respectively, abuts against one another which in turn causes the first abutting member to be captured inside the slot. Moving back the device to the first locking position by rotating the pin driver and the pin relative to each other in the first direction allows the pin to be readily pulled out of the pin driver.

### TECHNICAL FIELD

The present invention generally relates to surgical instruments and more particularly to a device for use in inserting a surgical pin into a bone structure.

### BACKGROUND

Maintaining proper anatomical alignment and angulations in surgical procedures often requires the use of surgical pins. Such surgical pins may be used as implants to stabilize broken bones or as instruments for stabilizing other surgical instruments. Surgical pins are of great value in performing, for example, a knee replacement procedure where a femoral cutting block is positioned on an anterior, distal portion of a femur adjacent the condyles of a knee for guiding an oscillating bone saw to cut the knee bone to fit a matching prosthesis. The positioning of the femoral cutting block should be accompanied by fixedly and stably holding the same in place. One or more surgical pins are usually driven into the knee's femur portion to prevent unnecessary movement of the femoral cutting block. The degree of stability of the femoral cutting block depends on the steadiness of the fixedly held surgical pins, i.e., the femoral cutting block is more stabilized if the surgical pins supporting it are properly disposed on the knee with regard to the holes of the femoral cutting block through which the surgical pins pass through.

In any fixing means such as that described above, the use of multiple surgical pins is typically preferred so as to fix the position of any surgical instrument in place. Most of the surgical pin drivers in the industry today are configured to receive and hold a single pin. Thus, in cases where, for example, four pins are required to fix a position of a femoral cutting block on a knee bone, the conventional pin drivers have to be used four times. This arrangement necessitates a surgeon to drive a first pin into the bone using the pin driver, withdraw the pin driver from its attachment to the first pin, reload the pin driver with a second pin, and then drive the second pin into the bone again. This loop of steps is continually repeated until such time that a fourth or the last pin is finally driven into the bone.

The step of reloading the pin driver with a subsequent pin by itself is time-consuming and usually introduces considerable delays and intricacies into the process of performing a bone surgery. Customarily, such pin driver of the type that is designed to hold and drive a single pin is used by a surgeon in such a manner that the surgeon is obliged to detach the pin driver from the pin to that is fixedly positioned on the bone.

Surgical pins can be driven into a bone structure with the use of surgical power drills. A typical surgical power drill usually includes a chuck or a similar device for holding the surgical pin. The surgical pin is attached to the chuck of the surgical power drill. The pin is then driven into the bone by rotating it using is the surgical power drill while exerting a downward pressure onto the surface of the bone. Although the chuck, such as that marketed by Jacob Chuck Company, is generally easy to use, attaching and detaching the surgical pin from the chuck of the power drill adds an extra step and can delay the surgical procedure, particularly if multiple pins have to be used.

U.S. Pat. No. 3,026,870, issued on Mar. 27, 1962 to Charles W. Buckingham, discloses a surgical pin driver that includes a cylindrical shank having a striking end and a longitudinal bore extending partially into the opposite end of the striking end. An adapter element in the form of a threaded stud has a flat portion formed at its one end for insertion into the longitudinal bore of the aforementioned cylindrical shank. There is also included in this pin driver a radially extending set screw carried by the cylindrical shank. The set screw releasably secures the adapter stud within the longitudinal bore. The actions of tightening and loosening the set screw included in the same pin driver introduce difficulties and significant delay in a surgical procedure.

In view of the limitations of the abovementioned prior art, a need therefore exists for providing a device suitable for use in inserting a pin into an s object wherein the device is simple in construction and allows a pin to be readily attached and detached from a pin driver so as to prevent delays in a surgical procedure.

### DISCLOSURE OF THE INVENTION

The present invention provides a device suitable for use in inserting a to pin into an object comprising: (1) an elongated pin driver having a cavity extending longitudinally from one end thereof, the cavity defining an interior wall member of the pin driver, and a first abutting member extending transversely of the cavity from the interior wall member of the pin driver, wherein the first abutting member has first and second abutment surfaces; (2) is an elongated pin for engagement with the pin driver, the pin having a second abutting member on one end thereof, wherein the second abutting member has a third abutment surface for abutment against the first abutment surface of the first abutting member; and (3) a locking mechanism for locking the engagement of the pin driver and the pin.

Preferably, the locking mechanism includes a stud portion projecting longitudinally from the second abutting member of the pin. The locking mechanism further includes a slot extending partially into the stud portion and transversely of a longitudinal length of the stud portion. The stud portion defines a fourth abutment surface while the slot defines a fifth abutment surface. Furthermore, the slot is in spaced apart relation with one end of the stud portion projecting away from the second abutting member of the pin. The slot is also provided by at least one rounded edge about which the first abutting member along with the pin driver pivots. This configuration enables movement of the device to a first locking position when the pin is inserted into the pin driver such that each of the first and third abutment surfaces abuts against one another and such that each of the second and fourth abutment surfaces abuts against one another, and by means of which, the pin is prevented from moving further into the cavity of the pin driver, the pin driver and the pin are prevented from rotating relative to each other in a first direction, and the pin is allowed to be readily pulled out of the pin driver. The same configuration further enables movement of the device to a second locking position when the pin driver and the pin are rotated relative to each other in a to second direction substantially opposite the first direction until each of the second and fifth abutment surfaces abuts against one another which in turn causes the first abutting member to be captured inside the slot, and by means of which, the pin is prevented from moving further into the cavity of the pin driver and from being pulled out of the pin driver. In order to move the device is to an unlocking position, the pin driver and the pin can be rotated relative to each other in the first direction until the device is moved back to the first locking position. This thereby allows the pin to be readily pulled out of the pin driver.

Preferably, each of the pin driver and the pin has a cylindrical shape. The third abutment surface associated with the second abutting member of the cylindrical pin defines a first end face having a first surface area and the end of the stud portion defines a second end face having a second surface area, wherein said second surface area associated with the second end face is smaller than the first surface area associated with the first end face.

Preferably, the first abutting member has a length that allows the surface area of the stud portion to pass through a portion of the cavity associated with the pin driver.

In one embodiment, the pin of the device is a surgical pin and the object is a bone structure through which the surgical pin can be driven. In another embodiment, the pin is a surgical drill bit. A typical surgical drill bit has one end that is provided with a conical tip for insertion into the bone structure. The other end of the drill bit opposite the conical tip can be arranged to have a structure similar to the stud portion as described above so that the drill bit can be engaged with the pin driver through the locking mechanism as described above.

For a better understanding of the invention and to show how the same may be performed and carried out into practice, a preferred embodiment will now be described, by way of non-limiting example only, with reference to the accompanying drawings.

### BRIEF DESCRIPTION OF THE DRAWINGS

FIG. 1 is an isometric view of a preferred embodiment of a device suitable for use in inserting a pin into an object according to the invention;

FIG. 1A is an enlarged fragmentary view of FIG. 1;

FIG. 2 is an exploded view of FIG. 1;

FIG. 3 is an enlarged fragmentary isometric view of FIG. 1, with parts broken away, showing a locking mechanism wherein a pin driver and a pin of the device are disengaged;

FIG. 4 is another enlarged fragmentary isometric view of FIG. 1, with parts broken away, showing a locking mechanism wherein the pin driver and the pin of the device are engaged;

FIG. 5 is an exploded fragmentary view of FIG. 4;

FIG. 6 is a fragmentary side view of FIG. 1;

FIG. 7 is a view of the device of FIG. 1 being handled by a user;

FIG. 8 is a view of the device of FIG. 1 being used in inserting a pin into an object using a surgical power drill; and

FIG. 9 is an enlarged, exploded fragmentary view of FIG. 8, with parts broken away, showing a locking mechanism wherein the pin driver and the pin of the device are engaged.

### DESCRIPTION OF PREFERRED EMBODIMENTS

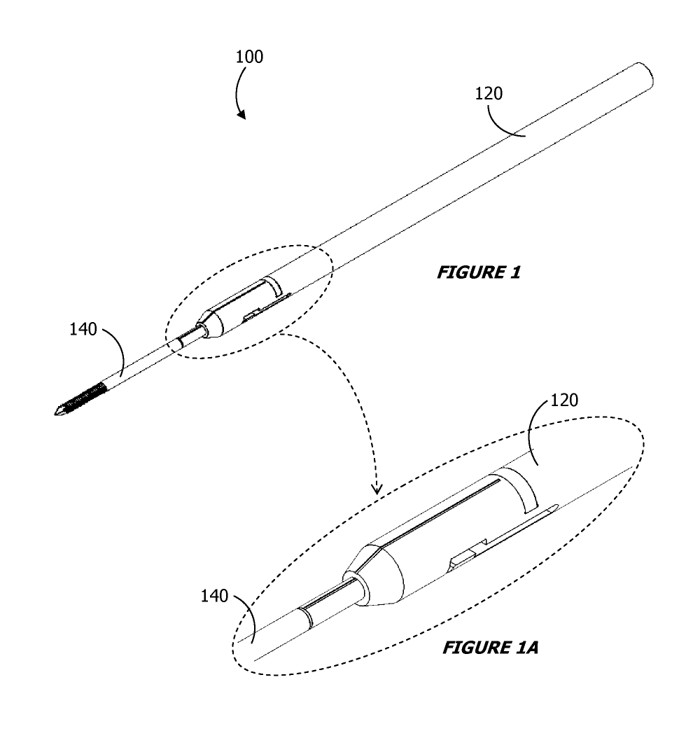

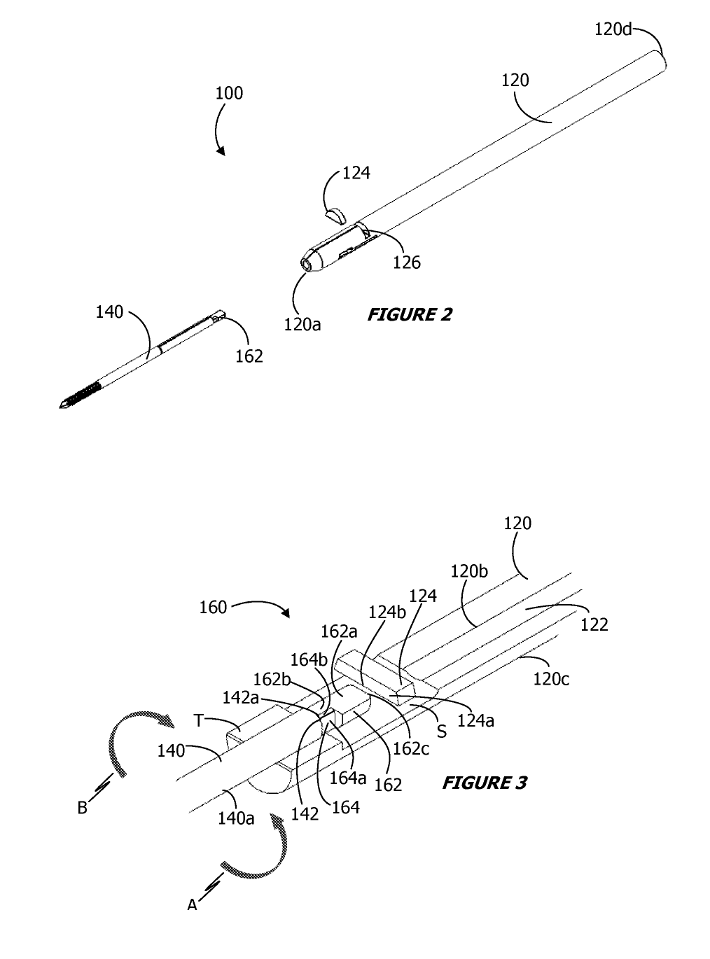

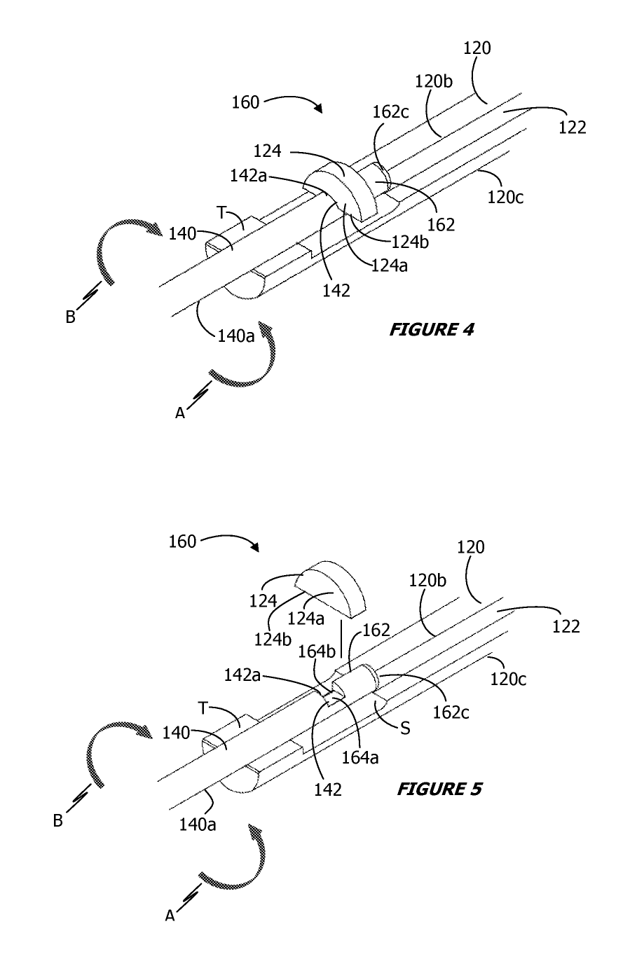

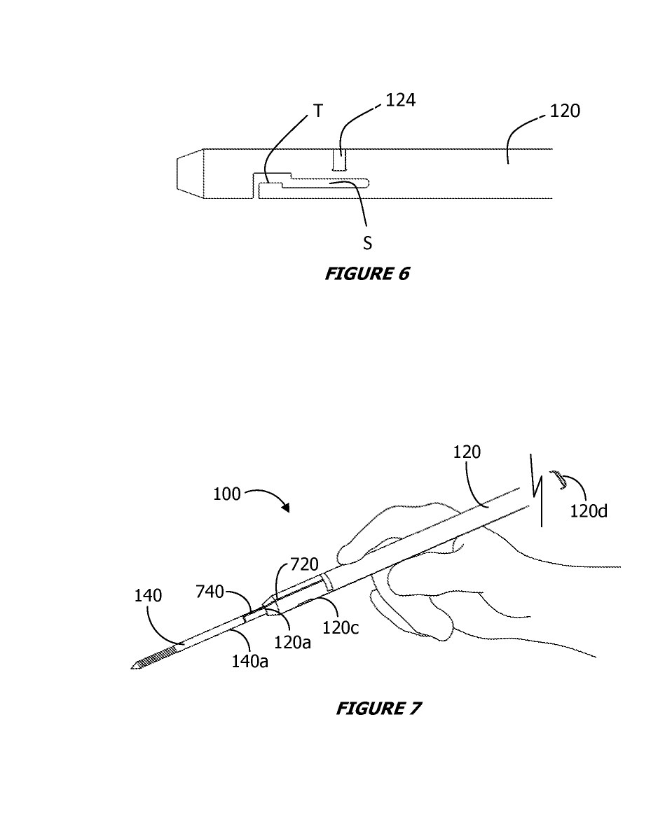

Referring to FIGS. 1, 1A, 2, 3, 4, 5, and 6, there are shown different views of a device suitable for use in inserting a pin into an object, generally to designated by reference numeral 100, according to a preferred embodiment of the present invention. In particular, FIG. 1 is an isometric view of the device 100, FIG. 1A is an enlarged fragmentary view of FIG. 1, and FIG. 2 is an exploded view of FIG. 1 while FIGS. 3 and 4 show enlarged fragmentary isometric views of FIG. 1 in which some parts of the device 100 are broken is away so as to illustrate the components of the device 100 of the present invention in a clear manner. FIG. 5 is an exploded fragmentary view of FIG. 4. FIG. 6 is a fragmentary side view of FIG. 1. The device 100 comprises mainly of an elongated pin driver 120 having a cavity 122 extending longitudinally from its one end 120a and a pin 140 for engagement with the pin driver 120 passing through the longitudinal cavity 122. The pin driver 120 has a cylindrical shape with a hollow interior defining the cavity 122. On the other hand, the pin 140 is shaped as a solid rod which likewise has a cylindrical cross-section. The cavity 122 defines an interior wall member 120b of the pin driver 120 and extends throughout the longitudinal axis of the pin driver 120. It is the interior wall member 120b of the pin driver 120 that comes in contact with an exterior wall member 140a of the pin 140 wherein said contact allows the pin driver 120 and the pin 140 to rotate relative to each other. The rotational movement of each of the pin driver 120 and the pin 140 relative to one another is simply effected by virtue of the sizes of the pin driver 120 and the pin 140. Limits and fits in mechanical engineering can be arranged desirably to achieve different types of fit for the contact of the interior wall member 120b associated with the pin driver 120 and the exterior wall member 140a associated with the pin 140. For example, the diameter of the interior wall member 120b associated with the cylindrical pin driver 120 can be arranged to make it slightly larger than the diameter of the exterior wall member 140a associated with the cylindrical pin 140 so as to produce a fit of, for example, sliding type or loose type.

The pin driver 120 further includes a first abutting member 124 extending transversely of its cavity 122 from its interior wall member 120b. The first abutting member 124 is shaped as a half-circle, and the outside diameter of said half circle-like first abutting member 124 substantially matches the diameter of an exterior wall member 120c associated with the cylindrical pin driver 120. The first abutting member 124 has a length defined by its radius that is less than one-half of the radius of the cylindrical pin driver 120. The first abutting member 124 is inserted into an opening 126 which is formed transversely of the cavity 122 from the exterior wall member 120c to the interior wall member 120b of the pin driver 120, and said opening 126 has a shape that substantially matches the shape of the first abutting member 124. Moreover, the first abutting member 124 has a first abutment surface 124a and a second abutment surface 124b, both of which are substantially flat. Particularly, the second abutment surface 124b is arranged to face towards the cavity 122. The first abutting member 124 can be fixed to the pin driver 120 either by welding or by using any suitable adhesive.

The pin 140 further includes a second abutting member 142. This second abutting member 142 has a third abutment surface 142a that is substantially flat. The pin 140 can be continuously inserted into the pin driver 120 through the cavity 122 of the pin driver 120 until the third abutment surface 142a abuts against the first abutment surface 124a of the first abutting member 124 provided into the opening 126 formed in the pin driver 120. With this configuration, the first abutting member 124 essentially serves as a stopper which stops the sliding motion of the pin 140 that is being passed through the cavity 122 of the pin driver 120. In other words, the abutment of the first and third abutment surfaces 124a, 142a is an indicator that the pin 140 can no longer be pushed further into the cavity 122 of the pin driver 120.

A locking mechanism 160 is associated with the pin driver 120 and the pin 140. More particularly, the locking mechanism 160 is designed for locking to an engagement of the pin driver 120 and the pin 140. The locking mechanism 160 includes a stud portion 162 that projects longitudinally from the second abutting member 142 of the pin 140. Similar to the shape of the pin 140, the stud portion 162 also depicts a cylindrical cross section. It means that the diameter of the exterior wall member 140a of the pin 140 is substantially the is same as the exterior diameter of the depicted cylindrical shape of the stud portion 162. However, the stud portion 162 is partially cut along its length producing flat portions 162a, 162b. Specifically, the flat portions 162b serves as a fourth abutment surface. The locking mechanism 160 further includes a slot 164 extending partially into the stud portion 162 and transversely of a longitudinal length of the stud portion 162. The slot 164 defines a flat portion 164a which serves as a fifth abutment surface. The position of the slot 164 is in spaced apart relation with one end 162c of the stud portion 162 projecting away from the second abutting member 142 of the pin 140.

The third abutment surface 142a associated with the second abutting member 142 of the pin 140 defines a first end face having a first surface area and the end 162c of the stud portion 162 defines a second end face having a second surface area. The second surface area associated with the end 162c of the stud portion 162 is smaller than the first surface area associated with the third abutment surface 142a. Furthermore, the first abutting member 124 has a length that allows the second surface area of the end 162c of the stud portion 162 to pass through a portion of the cavity 122 defined by the interior wall member 120b of the pin driver 120.

The pin driver 120 is further provided with a space (S) extending from its interior wall member 120b in the direction towards its exterior wall member 120c. The space (S) is clearly shown in FIG. 6. This space (S) is created by cutting a portion of the cylindrical pin driver 120 along a route from the exterior wall member 120c of the pin driver 120 to its interior wall member 120b that is in a sliding contact with the exterior wall member 140a of the pin 140 when the pin 140 is inserted into the pin driver 120. A portion between the interior wall member 120b and the exterior wall member 120c of the pin driver 120 left after the cut is characterized by a substantially thin material that can be subjected to slight deformation towards the cavity 122 during manufacturing of the pin driver 120. This deformation creates a bias of a tip (T) of the portion of the pin driver's interior and exterior wall members 120b, 120c left after the cut towards the cavity 122 which in turn results in an interference of said tip (T) with the exterior wall member 140a of the pin 140 when the pin 140 is inserted into the pin driver 120. This interference makes the portion of the pin driver's interior and exterior wall members 120b, 120c left after the cut act like a spring or clamp as if there is a finger pushing it towards the cavity 122 of the pin driver 120. In effect, the interference holds the pin 140 against the interior wall member 120b associated with the cavity 122 of the pin driver 120 by virtue of friction between them. The friction prevents the pin 140 from inadvertently falling off from the pin driver 120 once the pin 140 has been inserted into the pin driver 120. The friction which acts to maintain the engagement of the pin driver 120 and the pin 140 is particularly advantageous when the device 100 is in use, as the device 100 can be held by a surgeon in different positions without requiring any fasteners such as screws, bolts, or the like.

Referring particularly to FIGS. 3 and 4, both of which show an enlarged fragmentary isometric view of FIG. 1, illustrated is a locking mechanism 160 wherein the pin driver 120 and the pin 140 of the device 100 are disengaged and engaged, respectively. FIG. 5 is an exploded view of FIG. 4. In these figures, some parts are broken away in order to clearly illustrate how the locking mechanism 160 operates with regard to a preferred embodiment of the present invention. A first locking position is defined by the locking mechanism 160 wherein the pin 140 is inserted into pin driver 120 such that the third abutment surface 142a abuts against the first abutment surface 124a and the fourth abutment surface 162b abuts against the second abutment surface 124b, wherein the fourth abutment surface 162b is substantially parallel to the second abutment surface 124b. In this first locking position, the pin driver 120 and the pin 140 are prevented from rotating relative to each other in a first direction due to the abutment of the fourth abutment surface 162b against the second abutment surface 124b. This first direction characterizes a clockwise direction when the device 100 is viewed from an opposite end 120d of the end 120a of the pin driver 120 as clearly shown in FIG. 2. As shown in FIGS. 2, 3, and 4, for example, the pin driver 120 is prevented from rotating relative to the pin 140 in the direction indicated by arrow A while the pin 140 is prevented from rotating relative to the pin driver 120 in the direction indicated by arrow B. The slot 164 is adapted to have at least one rounded edge 164b about which the first abutting member 124 along with the pin driver 120 pivots. This allows the pin driver 120 and the pin 140 to rotate relative to each other in a second direction that is substantially opposite the first direction. This second direction characterizes a counter-clockwise direction when the device 100 is viewed from the opposite end 120d of the end 120a of the pin driver 120, wherein the opposite end 120d is clearly shown in FIG. 2. As shown in FIGS. 2, 3, and 4, for example, the pin driver 120 rotates relative to the pin 140 in the direction indicated by arrow B while the pin 140 rotates relative to the pin driver 120 in the direction indicated by arrow A. Thus, in the first locking position, the pin 140 is prevented from moving further into the cavity 122 of the pin driver 120 and, at the same time, the pin driver 120 and the pin 140 are prevented from rotating relative to each other in the direction indicated by arrow A (or in a clockwise direction when the device 100 is viewed from the opposite end 120d of the end 120a of the pin driver as clearly shown in FIG. 2) for the pin driver 120 and arrow B (or in a counter-clockwise direction when the device 100 is viewed from the opposite end 120d of the end 120a of the pin driver 120 as clearly shown in FIG. 2) for the pin 140. However, the first locking position allows the pin 140 to be pulled out of the pin driver 120. While the pin driver 120 and the pin 140 are in the first locking position, any of the pin driver 120 and pin 140 can be rotated to move the device 100 to a second locking position as described below. As shown in FIGS. 2, 3, and 4, for example, the second locking position is achieved when pin driver 120 is rotated relative to the pin 140 as indicated by arrow B or when the pin 140 is rotated relative to the pin driver 120 as indicated by arrow A.

After the pin 140 is positioned inside the pin driver 120 wherein the device 100 is moved to the first locking position, each of the pin driver 120 and the pin 140 can be rotated approximately 90 degrees following the directions indicated by arrows B and A, respectively, relative to one another until the second abutment surface 124b and the fifth abutment surface 164a abuts against one another as clearly shown in FIGS. 4 and 5. In this second locking position, the first abutting member 124 is captured inside the slot 164 thereby preventing the pin 140 from being pulled out of the pin driver 120. This defines a second locking position. In this second locking position, the pin 140 is prevented from moving further into the cavity 122 of the pin driver 120 while also being prevented from being pulled out of pin driver 120. At the same time, the pin driver 120 is prevented from rotating relative to the pin 140 in the second direction as indicated by arrow B while the pin 140 is prevented from rotating relative to the pin driver 120 in the first direction as indicated by arrow A. In order to move the device 100 to an unlocking position, the pin driver 120 can be rotated back approximately 90 degrees relative to the pin 140 following the first direction indicated by arrow A (or in a clockwise direction when the device 100 is viewed from the opposite end 120d of the end 120a of the pin driver 120, wherein the opposite end 120d is clearly shown in FIG. 2) thereby moving the device 100 from the second locking position back to the first locking position. Moving the device 100 from the second locking position back to the first locking position can also be achieved by rotating back the pin 140 approximately 90 degrees relative to the pin driver 120 following the second direction as indicated by arrow B (or in a counter-clockwise direction when the to device 100 is viewed from the opposite end 120d of the end 120a of the pin driver 120, wherein the opposite end 120d is clearly shown in FIG. 2). With the first locking position back in place, the pin driver 120 and the pin 140 can be readily disengaged from each other. In essence, the first locking position allows the pin 140 to be readily pulled out of the pin driver 120.

In use, the pin 140 can be inserted into the pin driver 120 in order to move the device 100 to the first locking position. A surgeon can then push and rotate the pin driver 120 following the first direction as indicated by arrow A (or in a clockwise direction when the device 100 is viewed from the opposite end 120d of the end 120a of the pin driver 120, wherein the opposite end 120d is clearly shown in FIG. 2) to drive the pin 140 into the object to be pinned such as, for example, a bone structure. Once the pin 140 is inserted into the object to the desired depth, the surgeon can simply pull the pin driver 120 away from the pin 140, and the pin 140 can be left attached to the object. To remove the pin 140 from the object, the pin driver 120 can be placed back over the pin 140 moving the device 100 to the first locking position. The surgeon can then rotate the pin driver 120 in the second direction as indicated by arrow B (or in a counter-clockwise direction when the device 100 is viewed from the opposite end 120d of the end 120a of the pin driver 120, wherein the opposite end 120d is clearly shown in FIG. 2) in order to move the device 100 to the second locking position. The surgeon can continue the rotation following the second direction as indicated by arrow B and, at any given point of time, pull the pin driver 120 away from the bone structure. Since the pin driver 120 and the pin 140 are in the second locking position, the pin 140 moves together with the pin driver 120 and then the pin 140 can be pulled out of the object. Since there are only two components involved in using the device 100, namely, the pin driver 120 and the surgical pin 140, the surgeon no longer needs to manipulate a third component or any additional number of components for that matter. The construction of the device 100 is simple, and the surgeon is therefore able to save a significant amount of time in installing the pin 140 into to the cavity 122 of the pin driver 120.

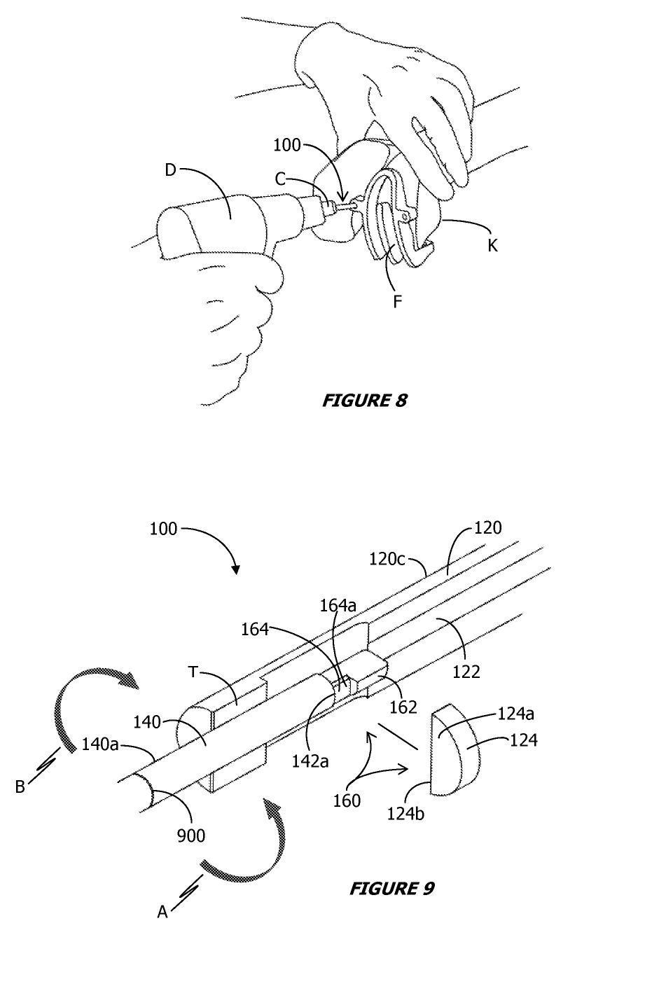

Referring now to FIGS. 7 and 8, there is shown the device 100 illustrated in FIG. 1 being handled by a user and being used in inserting the pin 140 into an object (K) using a surgical power drill, respectively. FIG. 9 is an enlarged, exploded fragmentary isometric view of FIG. 8, with parts is broken away, showing the locking mechanism 160 wherein the pin driver 120 and the pin 140 of the device 100 are engaged. Preferably, the device 100 is used in inserting a surgical pin 140 into a bone structure such as for example a knee (K). The device 100 may used manually by a surgeon. Alternatively, the device 100 can also be used in conjunction with a standard drilling tool (D) that is commonly used in surgical operations and is more commonly known as surgical power drill. At the discretion of the surgeon, the device 100 can be held by the surgeon who may apply an adequate force or specifically downward pressure in order to insert the surgical pin 140 into a substantially soft surface surrounding the knee bone (K). Where the surgical pin 140 has a threaded tip for insertion into the knee (K), the surgeon can drive the surgical pin 140 manually using the pin driver 120 in order to effect the insertion, or attach to the surgical power drill (D) to drive the pin 140 into the knee (K). Where the surgical pin 140 is of type that requires blowing in order to be driven into the knee (K), the surgeon may strike the end 120d of the pin driver 120 in order to force the insertion of the surgical pin 140 into the knee (K). In any case, the surgeon can simply prepare the pin driver 120 and the surgical pin 140. Before threading or impacting the surgical pin 140 into the knee (K) or surrounding portions thereof, the surgical pin 140 can be inserted into the pin driver 120 by the surgeon or by anyone assisting the surgeon. In this insertion process, it is highly advantageous that the pin driver 120 is provided with a marker section 720 which is arranged to be recognizable from the outside of its exterior wall member 120c. With a further marker section 740 provided on the exterior wall member 140a of the surgical pin 140 and which is also arranged to be recognizable from the outside of the exterior wall member 140a of the to surgical pin 140, it is possible that the marker section 720 on the pin driver 120 can be matched to the further marker section 740 on the surgical pin 140 such that a single straight line or path is formed. This straight line ensures that the surgical pin 140 is inserted into the pin driver 120 such that the device 100 is moved to the first locking position as described above. In another embodiment, is the pin 140 is a surgical drill bit. A typical surgical drill bit has one end that is provided with a conical tip for insertion into the bone structure. The other end of the drill bit opposite the conical tip can be arranged to have a structure similar to the stud portion 162 as described above so that the drill bit can be engaged with the pin driver 120 through the locking mechanism 160 as described above.

In an instance where for example a femoral cutting block (F) is required to be installed during a standard Total Knee Arthroplasty (TKA) procedure, multiple surgical pins 140 are also necessary to be used in order to secure the femoral cutting block (F) in place before a surgeon starts cutting any portion of the knee (K). Since only the pin driver 120 and the surgical pin 140 are as fewest components that are required to be manipulated by the surgeon, the surgeon is able to save a considerable amount of time in installing more than one surgical pins 140. The locking mechanism 160 of the device 100 allows each of the surgical pins 140 to be readily detached from the pin driver 120 thereby preventing delays in the surgical procedure. Another advantage of the device 100 of the present invention is that the risk of contaminating the surgical pins 140 is minimized. Since each of the surgical pins 140 is securely engaged within the cavity 122 of the pin driver 120 by the tip (T) at all times, there is a low tendency that the surgical pin 140 would inadvertently fall off to the ground. One notable advantage of the present invention becomes apparent when the device 100 is used with a surgical power drill (D), wherein the pin driver 120 of the device 100 is inserted to a chuck (C) of the drill (D) and then use the drill (D) to insert the pin 140 into the knee bone (K). Particularly, the end 120d of the pin driver 120 is the portion of the device 100 to that can be inserted into the chuck (C). Once a first pin 140 is drilled into the knee bone (K) using the first locking position as described above, the surgeon performing the drilling procedure can conveniently disengage the pin driver 120 from the drilled pin 140 and replace it with another pin 140 without the need to tighten or loosen the chuck (C). While a surgical procedure is ongoing, the pin is driver 120 can be left held in the drill's chuck (C). In this manner, the surgeon performing the drilling operation is able to save time by spontaneously feeding a pin 140 into the pin driver 120 every after a successful insertion of the pin 140 is accomplished. This means that the surgeon no longer needs an additional step to manually fix any component in the process of drilling since all that is required is to drill the pin 140 into the knee bone (K) and once the desired depth of insertion is achieved, the drill (D) carrying the pin driver 120 can be withdrawn smoothly and completely from its attachment to the pin 140 driven into the knee bone (K). Thereafter, a succeeding pin 140 can be pushed into the cavity 122 of the pin driver 120. To remove a pin 140 from the knee bone (K), the pin driver is simply placed over the pin 140 until the device 100 is moved to the first locking position wherein the pin 140 is placed inside the pin driver 120. The surgeon then rotates the drill (D) carrying the pin driver 120 in the direction indicated by arrow B in FIG. 9 to move the device 100 to the second locking position. In this position, the fifth abutment surface 164a associated with the slot 164 provided in the stud portion 162 of the pin 140 abuts against the second abutment surface 124b. The surgeon continues to the rotate the drill in the direction indicated by arrow B in FIG. 9 and, at any given point of time, pull the drill (D) away from the knee (K) to remove the pin 140 from the knee bone (K).

Although it is described from the above disclosure that there are marker and further marker sections 720, 740 provided on the pin driver 120 and the pin 140, respectively, to enable insertion of the pin 140 into the pin driver 120 in a proper position, i.e., the first locking position as described above wherein the first abutment surface 124a of the first abutting member 124 abuts against the third abutment surface 142a and wherein the second abutment surface to 124b of the first abutting member 124 abuts against the fourth abutment surface 164a of the slot 164, an alternative marker section 900 around a circumference of the pin 140 can also be made. Such an alternative marker section 900 around the circumference of the pin 140 serves as an indicator that the pin 140 is already in the proper position inside the cavity 122 of the pin is driver 120. The circumferential alternative marker section 900 reaching the end 120a (as clearly shown in FIG. 2) of the pin driver 120 is an indicator that the pin 140 has been set in place through the cavity 122 of the pin driver 120. Each of the marker section 720, further marker section 740, and alternative marker section 900 is preferably made by laser marking or machining the exterior wall members 120c, 140a, of the pin driver 120 and the surgical pin 140, respectively.

The pin driver 120, the surgical pin 140, and the components associated with the locking mechanism 160, as illustrated in previous figures, associated with the pin driver 120 and the surgical pin 140 are preferably made from stainless steel so that corrosion is prevented. One possible way to manufacture the device 100 is by using a wire saw or any suitable machine that utilizes a metal wire in performing a manual cut. It is likewise possible that the required cutting process is carried out automatically by Wire EDM (electrical discharge machining) cutting machine which utilizes an electrically energized thin wire to perform a cut. Such EDM cutting is suitable for mass production of the device 100 since the same may be operated with controlled parameters to effect rapid and consistent cut.

1. A device suitable for use in inserting a pin into an object comprising:
an elongated pin driver having
a cavity extending longitudinally from one end thereof, the cavity defining an interior wall member of the pin driver, and
a first abutting member extending transversely of the cavity from the interior wall member of the pin driver, the first abutting member having a first abutment surface and a second abutment surface;
an elongated pin for engagement with the pin driver, the pin having
a second abutting member on one end thereof, the second abutting member having a third abutment surface for abutment against the first abutment surface of the first abutting member; and
a locking mechanism for locking the engagement of the pin driver and the pin, the locking mechanism including
a stud portion projecting longitudinally from the second abutting member of the pin, the stud portion defining a fourth abutment surface, and
a slot extending partially into the stud portion and transversely of a longitudinal length of the stud portion, the slot being in spaced apart relation with one end of the stud portion projecting away from the second abutting member of the pin, the slot having at least one rounded edge about which the first abutting member along with the pin driver pivots, the slot defining a fifth abutment surface,
wherein the device is moved to a first locking position when the pin is inserted into the pin driver such that each of the first and third abutment surfaces abuts against one another and such that each of the second and fourth abutment surfaces abuts against one another, whereby the pin is prevented from moving further into the cavity of the pin driver, the pin driver and the pin are prevented from rotating relative to each other in a first direction, and the pin is allowed to be readily pulled out of the pin driver,
wherein the device is moved to a second locking position when the pin driver and the pin are rotated relative to each other in a second direction substantially opposite the first direction until each of the second and fifth abutment surfaces abuts against one another thereby causing the first abutting member to be captured inside the slot, whereby the pin is prevented from rotating further relative to the pin driver in the second direction, and the pin is prevented from moving further into the cavity of the pin driver and from being pulled out of the pin driver, and
wherein the device is moved to an unlocking position when the pin driver and the pin are rotated relative to each other in the first direction until the device is moved from the second locking position back to the first locking position thereby causing the first abutting member to be removed from the slot, and when the pin is pulled out of the pin driver.

2. The device according claim 1, wherein each of the pin driver and the pin has a cylindrical shape.

3. The device according to claim 1, wherein each of the pin driver and pin has a marker section and a further marker section, respectively, each of the marker section and the further marker section being arranged to be recognizable from the outside of exterior wall members of the pin driver and the pin, respectively.

4. The device according to claim 1, wherein the pin has an alternative marker section disposed around a circumference thereof, the alternative marker section being arranged to be recognizable from the outside of an exterior wall member of the pin.

5. The device according to claim 1, wherein the pin is a surgical drill bit.
